(19) United States
(12) Patent Application Publication
(10) Pub. No.: US 2008/0172185 A1
(43) Pub. Date: Jul. 17, 2008

(54) AUTOMATIC CLASSIFYING METHOD, DEVICE AND SYSTEM FOR FLOW CYTOMETRY

(75) Inventors: Hanping Yi, Shenzhen (CN); Jianjun Chu, Shenzhen (CN); Wen Gu, Shenzhen (CN)

Correspondence Address: Mindray c/o Bingham McCutchen LLP, Three Embarcadero Center, San Francisco, CA, 94111-4067, US

(73) Assignee: SHENZHEN MINDRAY BIO-MEDICAL ELECTRONICS CO., LTD., Shenzhen (CN)

(21) Appl. No.: 11/966,703

(22) Filed: Dec. 28, 2007

(30) Foreign Application Priority Data
Jan. 17, 2007 (CN) 200710072878.6

(51) Int. Cl. G01N 33/48

(52) U.S. Cl. 702/21

(57) ABSTRACT

An automatic classification method for flow cytometry includes characterizing cells or particles as a vector based on at least two-path optical signals generated when the cells or particles pass through an irradiated area one by one, calculating a distance between the effective cells or particles, in which a shorter distance indicates a higher similarity between the two cells or particles, clustering the cells or particles with high similarity into the same class, continuing to cluster similar cells or particles into the same class until the effective cells or particles are clustered into a number L of classes which should be contained in a sample and is determined based on a measuring principle. This method automatically classifies particles accurately and efficiently.

### RELATED APPLICATIONS

The present application claims priority to Chinese Patent Application No. 200710072878.6, filed Jan. 17, 2007, which is hereby incorporated by reference herein in its entirety.

### TECHNICAL FIELD

The present disclosure relates to a classification method, device and system for cells or particles.

### SUMMARY

The present disclosure provides a classification method, device and system for flow cytometry, which automatically classify cells or particles accurately and efficiently.

### BRIEF DESCRIPTION OF THE DRAWINGS

FIG. 1 is a schematic diagram illustrating signal paths for flow cytometry.

FIG. 2 is a schematic diagram illustrating a classification with respect to a two-dimensional scatter diagram.

FIG. 3 is a schematic diagram illustrating defects existing in conventional flow cytometry systems.

FIG. 4 is a structural block diagram of a system according to one embodiment.

FIG. 5 is a flow chart of a method according to one embodiment.

FIGS. 6a, 6b and 6c are schematic diagrams illustrating a process of “gating” according to one embodiment.

FIG. 7 is a flow chart of one embodiment of a clustering step in the method shown in FIG. 5.

FIG. 8 is a structural block diagram of one embodiment of a processor for classification and statistics shown in FIG. 4.

FIGS. 9 and 10 show classification results of two different samples using a method according to one embodiment.

FIGS. 11 and 12 show different classification results using the same fixed borderline according to one embodiment.

### DETAILED DESCRIPTION

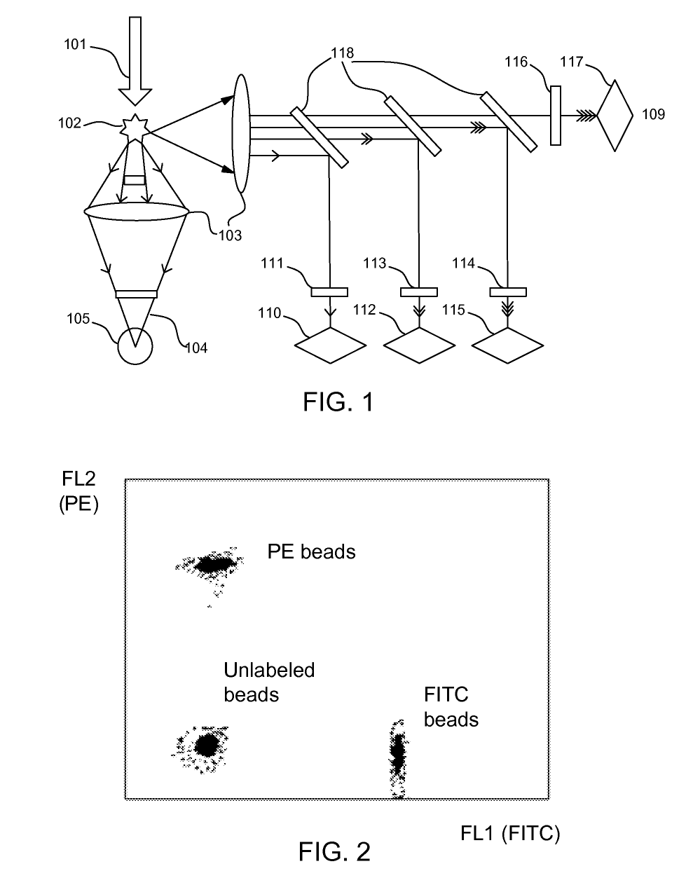

A flow cytometer, as well as a blood analyzer, a urine analyzer, a particle analyzer, etc., which are all based on flow cytometry, identifies different particles in a liquid and arranges them under different categories by collecting and analyzing two-dimensional or multidimensional data on the particles. As shown in FIG. 1, in a flow cytometer, cells or particles 102 encased by a sheath fluid pass one by one through an irradiated area where a particle 102 is irradiated by a laser 101 to generate different optical signals, such as a forward scattered signal (FSC) 104, a side scattered signal (SSC) 106 and multiple fluorescence signals (FL). For example, in FIG. 1, the reference numeral 107 represents a green fluorescence signal (FL1), the reference numeral 108 represents a yellow fluorescence signal (FL2), and the reference numeral 109 represents a red fluorescence signal (FL3). Convex lenses 103 (two shown) converge these optical signals. Additional optics, such as beam splitters 118 (three shown), may also be used to direct the signals to various detectors. A photodiode 105 detects the forward scattered signal 104. Bandpass filters 111, 113, 114, 116 and photomultipliers 110, 112, 115, 117 detect the side scattered signal 106, the green fluorescence 107, the yellow fluorescence 108 and the red fluorescence 109, respectively.

An analytic system (not shown) generates a two-dimensional or three-dimensional scatter diagram from the detected signals, into which a plurality of regions are divided. Particles with parameters that fall in the same region as one another are classified as the same class as one another. Thereafter, the number and percentage of particles belonging to the same class are calculated so as to analyze the statistical characteristics of the measured sample, as shown in FIG. 2.

According to a conventional method, classification is carried out using fixed borderlines in the scatter diagram. However, the fixed borderlines can only reflect the characteristics of a majority of normal samples. A shortcoming of this method is that it cannot adjust the borderlines for different samples. Thus, errors generally occur when the particle signal characteristics of a sample differ significantly from those reflected by the fixed borderlines. For example, U.S. Pat. No. 4,987,086 provides a method for distinguishing a neutrophil, a monocyte and a lymphocyte from a whole blood cell using “gating” in a scatter diagram of forward scattered light versus side scattered light. The so-called “gating” is to divide borderlines in the scatter diagram, and the cells that fall inside a certain borderline are considered as the same class. U.S. Pat. Nos. 4,727,020, 4,704,891, 4,599,307, 4,987,086 and 6,014,904 provide methods for identifying, classifying and counting cells in a blood sample by “gating.”

Pre-dividing the scatter diagram by borderlines may generate different regions representing different classes of particles, but these discrete regions may sometimes overlap such that particles that fall in the overlapped region may be incorrectly identified and classified. U.S. Pat. No. 5,627,040 uses a “gravitational attractor” to address this problem. Generally, this method uses borderlines, whose size, shape and azimuth (except for the positions) are fixed, for classification with respect to a scatter diagram, and then uses an optimum algorithm to determine the position of the borderline of each class based on the gravitational attractor of each class.

However, although the position of the borderline can be automatically adjusted by the above-mentioned “gravitational attractor” method, its size, shape and azimuth are still fixed. The problem of addressing individual differences among samples remains unsolved when the particles, especially human blood cells, are classified using the above-mentioned fixed borderlines. That is to say, the fixed borderlines are only effective for examining general characteristics of a majority of samples, but are incorrect in the case of human blood samples because there are individual differences. For example, after being treated by a reagent, the monocytes and lymphocytes of some people become larger. Errors occur if the general “fixed borderline” classification is used in such circumstances.

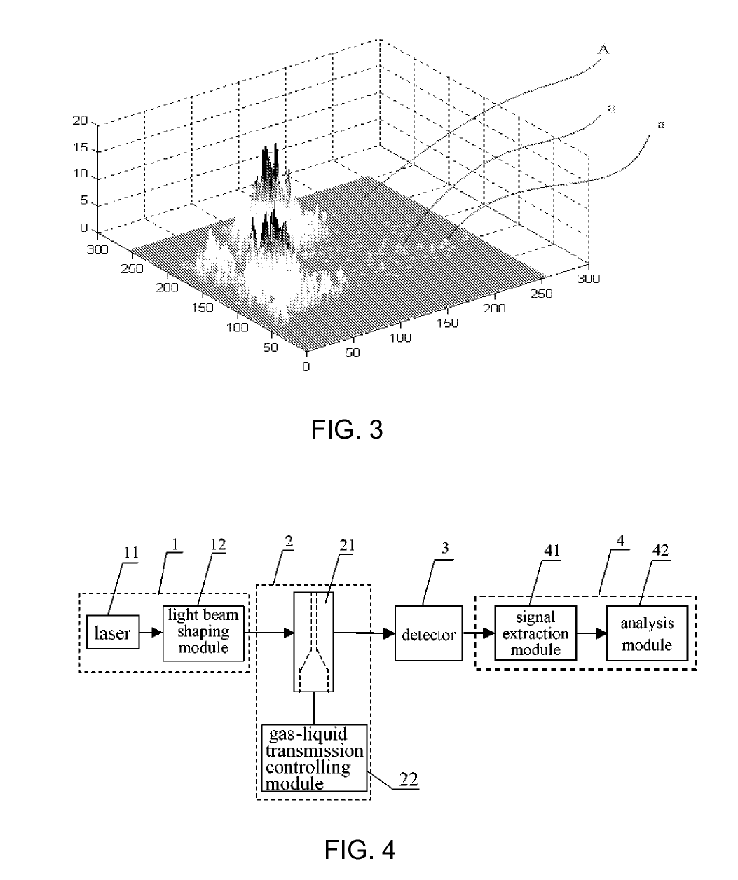

In the case of such individual differences, the general solution is to reposition/relocate the borderlines manually in the scatter diagram, which results in reduced efficiency. Therefore, this solution is not suitable for a supermatic instrument. U.S. Pat. No. 6,944,338 provides an automatic classification method, which uses a modified Koonst and Fukunaga algorithm to locate borderlines for the two-dimensional data (i.e., the wave troughs of two-dimensional data), and classifies the particles that fall into the same region formed by certain borderlines into the same group. However, there are also shortcomings in this method. For example, because there are discontinuities among data points in the scatter diagram, there is no data for many single points or small clusters of points, such as the region “a” shown in FIG. 3. According to this algorithm, the process of locating borderlines is performed around these points, and these points may eventually be classified as a separate class, respectively. However, these points do not belong to a separate class, but include particles that belong to a major class that are spaced farther apart from the others. Another shortcoming of this method is that it is difficult to address the above-mentioned problem, even if bins are used to smooth data. Rather, when the data is given further smoothing (i.e., more points in each bin), a larger deviation occurs when the calculated wave trough is converted to the original data. Yet another shortcoming of this algorithm is that it involves each point in the two-dimensional scatter diagram. However, there is generally only a small number of points that are effective in the two-dimensional scatter diagram, and no data may be obtained with respect to a plurality of regions, such as the region “A” shown in FIG. 3. The two-dimensional scatter diagram is generally a sparse matrix. Thus, the efficiency of the algorithm is reduced if each point is scanned.

Therefore, an automatic classification method, device and system for a flow cytometry are provided in the present disclosure, which automatically classify the particles accurately and efficiently. In one embodiment, there is provided an automatic classification method for a flow cytometry. The method includes characterizing cells or particles as a vector that is at least two-dimensional and associated with an intensity of optical signals in various paths thereof, based on at least two-path optical signals generated when the cells or particles are passing through an irradiated area one by one. The method further includes calculating a distance between the cells or particles, in which a shorter distance indicates higher similarity between the two cells or particles. The method further includes clustering the cells or particles with high similarity into the same class until the effective cells or particles are clustered into a number L of classes, which should be contained in a sample and is determined based on a measuring principle.

In certain embodiments, the method further includes setting a threshold to delete data of the cells or particles that do not meet the criterion of the threshold. The effective cells or particles may be finally clustered into one class.

The method may further include evaluating the clustering effect to determine a correct number of classes that should be contained in a sample. The evaluation may include calculating parameters about the clustering effect corresponding to integers from 1 to L+r respectively, where L is a number of classes that should be contained in a sample and determined based on the measuring principle, and is an integer larger than or equal to 1, and wherein r is an empirically determined integer larger than 0. The determination may also include locating an integer q corresponding to the biggest parameter about the clustering effect, and comparing the integer q with the number L of classes. If q>L, the number of classes in the sample is q. If L−o<q≦L, L is the number of classes in the sample. If q≦L−o, classification and calculation terminate.

According another embodiment, an automatic classification device for flow cytometry includes an event generation unit for characterizing cells or particles as a vector that is at least two-dimensional and associated with the intensity of optical signals in various paths based on at least two-path optical signals generated when the cells or particles are passing through an irradiated area one by one. The device also includes a calculation unit for calculating a distance between every two cells or particles based on the vector generated by the event generation unit, in which a shorter distance indicates a higher degree of similarity between two cells or particles. The device also includes a clustering unit for clustering the cells or particles with high similarity into the same class, which is operable to repeat clustering for multiple times until at least the effective cells or particles are clustered into a number L of classes that should be contained in a sample based on a measuring principle.

In certain embodiments, the automatic classification device further includes a gating unit for setting a threshold to delete data of the cells or particles that do not meet the criterion of the threshold. In addition, or in other embodiments, the clustering unit finally clusters the effective cells or particles into one class.

In certain embodiments, the device further includes a classification evaluation unit for evaluating a clustering effect to determine a correct number of classes that should be contained in a sample. The classification evaluation unit includes a second calculation module for calculating parameters about the clustering effects corresponding to integers from 1 to L+r respectively, where L is a number of classes that should be contained in a sample and is determined based on the measuring principle, and is an integer larger than or equal to 1, and r is an empirically determined integer larger than 0. A second locating module locates an integer q corresponding to the biggest parameter about the clustering effect, and a comparing module compares the integer q located by the second locating module with the number L of classes. If q>L, q is the number of classes in the sample. If L−o<q≦L, L is the number of classes in the sample. If q≦L−o, classification and calculation terminate.

According to another embodiment, an automatic classification and statistics system for a flow cytometry includes a sample generation device, including a gas-liquid transmission controlling module and a flow chamber, which are connected with each other. The gas-liquid transmission controlling module passes a sample fluid containing cells or particles to be measured and encased by a sheath of fluid through the flow chamber. The system also includes an irradiation device for emitting a light beam to irradiate the sheath fluid passing through the flow chamber, a detector for collecting at least two-path optical signals generated when the cells or particles are passing through an irradiated area one by one, and a processor for classification and statistics. The processor characterizes the cells or particles as a vector that is at least two-dimensional and associated with intensity of optical signals in various paths thereof based on the optical signals collected by the detector. It then calculates a distance between the effective cells or particles, in which a shorter distance indicates a higher degree of similarity between two cells or particles, and clusters the cells or particles with high similarity into the same class for multiple times until at least all of the effective cells or particles are clustered into a number L of classes that should be contained in a sample and is determined based on a measuring principle.

In certain embodiments, the processor for classification and statistics also sets a threshold before calculating the distance between the cells or particles to delete any data of the cells or particles that do not meet the criterion of the threshold. Further, the processor for classification and statistics may finally cluster all effective cells or particles into one class.

In some embodiments, the processor for classification and statistics further calculates parameters about the clustering effects corresponding to integers from 1 to L+r, locate an integer q corresponding to the biggest parameter about the clustering effect, and compares the located integer q with the number L of classes, wherein if q>L, q is the number of classes in the sample; if L−o<q≦L, L is the number of classes in the sample; and if q≦L−o, classification and calculation terminate, where L denotes a number of classes that should be contained in the sample and is determined based on the measuring principle, and is an integer larger than or equal to 1, and r denotes an empirically determined integer larger than 0.

The method or device according to embodiments of the present disclosure clusters particles into a certain class by analyzing and processing a collection of two-dimensional or multidimensional data concerning all particles to be measured. This method is based on data analysis, but not a borderline in a diagram (such as a one-dimensional histogram or two-dimensional scatter diagram). Thus it can apply to multidimensional data. According to the present method or device, data analysis, classification and counting are performed on each measured sample. This means that the borderlines for classification generated by this automatic clustering method vary with different samples. Therefore, the defect caused by fixed borderlines in classification can be overcome. That is, the present method or device can adjust the borderlines based on the specificity of the measured sample. Meanwhile, the classification method or device according to embodiments of the present disclosure calculates data coming from particles only and ignores the location where there is no particle. Thus the present method or device overcomes the defect associated with the Koonst and Fukunaga algorithm, according to which wave troughs are located based on discrete data, therefore improving efficiency of classification.

The present method or device also deletes unqualified data by establishing a gate before classification, which further reduces the amount of calculation and improves the efficiency of classification. Further, the present method or device evaluates classification effects after classification, which increases the credibility of the classification result, thus improving the accuracy of classification and the statistics of the particles.

A method described in the present embodiment is applicable to a flow cytometer as well as a blood analyzer, a urine analyzer and other particle analyzers that are based on a flow cytometry. According to the method, collection of two-dimensional or multidimensional data of the particles is analyzed and processed to classify the particles into respective classes that should be contained in a sample.

FIG. 4 shows a general classification and statistics system based on a flow cytometry according to one embodiment. The system includes a sample generation device 2, an irradiation device 1, a detector 3, and a processor for classification and statistics 4. The sample generation device 2 includes a gas-liquid transmission controlling module 22 and a flow chamber 21, which are connected with each other. The gas-liquid transmission controlling module 22 passes the sample fluid containing the cells or particles encased by a sheath fluid through the flow chamber 21. The flow chamber 21 according to one embodiment is a transparent part, including therein a square lead hole, through which the cells or particles encased by the sheath fluid pass one by one to be irradiated by a light beam. The irradiation device 1 emits a light beam to irradiate the sheath fluid passing through the flow chamber 21. The irradiation device 1 may include one or more laser sources 11 with different wavelengths and a beam shaping module 12 for shaping scattered light into a desired light beam. After passing through the beam shaping module 12, the light beam forms a spot at the lead hole of the flow chamber 21.

A variety of optical signals are generated when the sample fluid containing the measured cells or particles encased by sheath fluid passes through the spot. At least two-way optical signals are generally generated, such as a forward scattered signal (FSC), a side scattered signal (SSC) and multipath fluorescence signals (FL), as shown in FIG. 1. The detector 3 collects the at least two-way optical signals generated when the cells or particles pass through the irradiated area one by one. The detector 3 may be a photomultiplier (PMT) or photodiode (PD).

The processor for classification and statistics 4 characterizes each cell or particle as a vector that is at least two-dimensional and associated with the intensity of optical signals in various paths based on the optical signals collected by the detector 3, and also calculates a distance between effective cells or particles. The shorter the distance, the higher the degree of similarity between two cells or particles. The cells or particles with a high degree of similarity are clustered into the same class. After clustering multiple times, at least the effective cells or particles are allocated into a proper number L of classes that should be contained in a sample and that are determined based on a measuring principle.

In one embodiment, the processor for classification and statistics 4 includes a signal extraction module 41 and an analysis module 42. The signal extraction module 41 extracts the optical signals in each path collected by the detector 3. The analysis module 42 classifies the cells or particles based on their respective optical signals, and counts the cells or particles in each class.

According to one embodiment of flow cytometry, when a particle passes through a photo-induced area, two-dimensional or multidimensional signals concerning that particle may be acquired for characterizing that particle. The procedure starting from passing the particle through the photo-induced area to the acquisition of signals may be referred to as an event. If an instrument has a p-dimensional signal path, a p-dimensional vector eᵢ=(xᵢ₁i, xᵢ₂, xᵢ₃, . . . , xᵢₚ) can be obtained when the i^{th} particle passes through the irradiated area to trigger the event eᵢ, where xᵢₖ indicates intensity of the k^{th} signal. These signals are generally forward scattered signals (FSC), side scattered signals (SSC) or multipath fluorescence signals (FL1, FL2, . . . ). When n particles pass through in one measurement process, n events are triggered, thus obtaining data 1,

$${I}_{N \times P} = [ \begin{matrix}{x}_{11} & {x}_{12} & ⋯ & {x}_{1 p} \\ {x}_{21} & {x}_{22} & ⋯ & {x}_{2 p} \\ ⋮ & ⋮ & ⋮ & ⋮ \\ {x}_{n 1} & x - n 2 & ⋯ & {x}_{np}\end{matrix} ] .$$

The method according to the present embodiment analyzes and processes the data I and classifies all events in one measurement process into the desired classes.

In one embodiment, a method for classifying cells or particles using the analysis module 42 includes deleting invalid data to reduce the amount of calculation. Among the n events triggered in each measurement process, some events are not triggered by particles being examined. The number of these invalid events is sometimes huge and even greater than that of the valid events, which therefore increases the overhead of the calculation. Therefore, data concerning these invalid events is removed from the original data S to obtain data I_{m×p} corresponding to m valid events.

The invalid events generally come from fragments and noises generated by the reaction of particles and reagent, and have rather significant signal characteristics. Generally, they can be removed by “gating” via hardware or software. Gating includes setting a threshold, retaining the data falling within the threshold, and removing the data exceeding the threshold. Gating also includes an opposite process, i.e., removing the data falling within the threshold, and retaining the data exceeding the threshold. For two-dimensional data, gating includes setting a region. The data within the region are retained, and the data outside the region are removed, and vice versa.

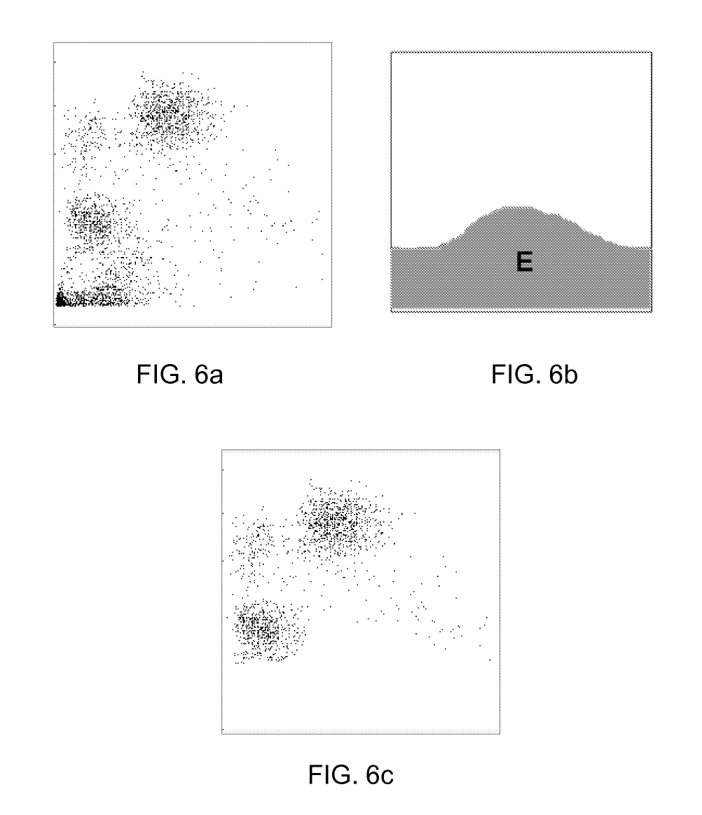

FIGS. 6a, 6b and 6c show an embodiment of removing invalid two-dimensional data according to one embodiment. In FIG. 6b, the region “E” may be considered as a “gate.” The data falling within this “gate” is deleted and does not participate in the clustering any longer, which may reduce the size of the calculation and improve the efficiency of the calculation. The region where an invalid event occurs in FIG. 6a is generally the region E in FIG. 6b. After an event k is triggered, data concerning this event is first examined. If (xₖ₁,xₖ₂)ε E, this event is considered an invalid event and the k^{th} data is removed to obtain effective data I_{m×p} with a relatively small volume (as shown in FIG. 6c).

The method for classifying cells or particles using the analysis module 42 may also include performing an analysis on the clustering of the effective data. Distances between events are calculated for determining the degree of similarity between the events. The shorter the distance, the higher the degree of similarity between two cells or particles.

Supposing d(eᵢ, eⱼ) represents the distance between events eᵢ and eⱼ, the following conditions should generally be satisfied:

d(eᵢ, eⱼ)≧0, and only when ei=ej, d(ei, ej)=0; (a)

d(eᵢ, eⱼ)=d(eⱼ, eᵢ); and (b)

d(eᵢ,eⱼ)≦d(eᵢ,eₖ)+d(eₖ,eⱼ). (c)

The total number of invalid events is generally a few thousand, (and generally less than ten thousand). The events that have data with the same characteristic are referred to as one event. That is, for two events eᵢ(xᵢ₁, xᵢ₂ . . . xᵢₚ) and eⱼ(xⱼ₁, xⱼ₂, . . . , xⱼₚ), if eᵢ=eⱼ, then only one of them participates in the clustering calculation, but the number of events is still counted as two. In this way, the amount of data is reduced, and calculation efficiency is enhanced.

The similarity may be examined based on various distances such as Euclidean distance, absolute distance, Minkowski distance, Chebyshev distance, weighted variance distance, Markov distance, etc. A proper distance may be selected based on the classification effect. In the following example, the similarity is examined in terms of Euclidean distance. The Euclidean distance between eᵢ and eⱼ is expressed as follows:

$$d ( {e}_{i} , {e}_{j} ) = {[ {\sum }_{k = 1}^{p} {( {x}_{ik} - {x}_{jk} )}^{2} ]}^{1 / 2} .$$

The distances between two events are calculated to form a collection of distances, for example, a distance matrix D_{m×m}

$${D}_{( 0 )} = [ \begin{matrix}0 & {d}_{12} & ⋯ & {d}_{1 m} \\ {d}_{21} & 0 & ⋯ & {d}_{2 m} \\ ⋮ & ⋮ & & ⋮ \\ {d}_{m 1} & {d}_{m 2} & ⋯ & 0\end{matrix} ] .$$

Cells or particles with a high degree of similarity are clustered into the same class. After multiple times of clustering, the effective cells or particles are allocated to a proper number L of classes contained in the sample that is determined according to a measuring principle. Meanwhile, each clustering is assigned a number, and the distances between classes are recorded during the course of clustering.

In general, when a sample is measured based on flow cytometry, it is possible to know in advance how many classes of particles that sample should have under a certain measuring principle. For example, there are about four or five subclasses of white blood cells when using a blood cell analyzer to classify and count the white blood cells. If a sample is known to have a number g of classes, the hierarchical diagram obtained by the above-mentioned method is only divided into g classes.

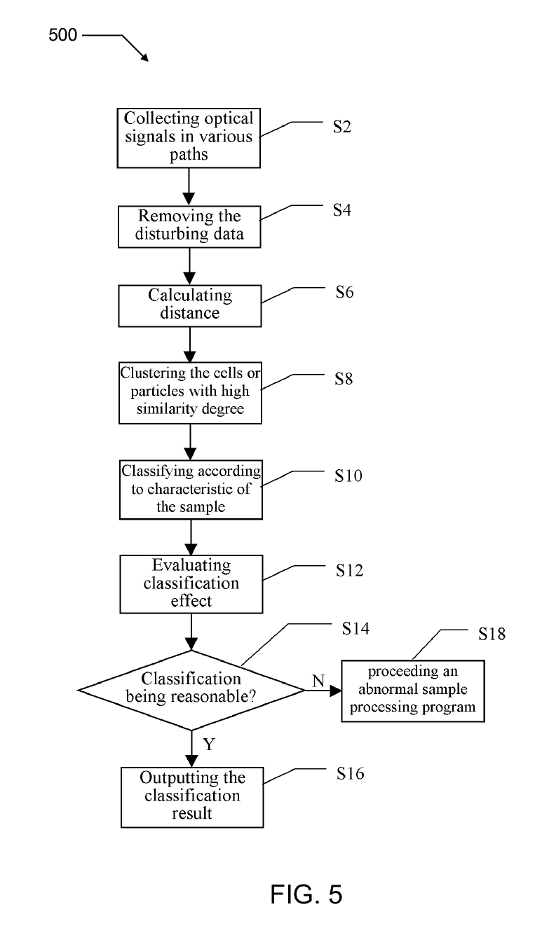

FIG. 5 shows another embodiment of a method 500 for classifying cells or particles by the analysis module 42. The method includes collecting S2 optical signals in various paths concerning cells or particles. Each measured cell or particle is characterized as a vector that is at least two-dimensional and associated with the intensity of the optical signals in various paths thereof. Thereafter, the cells or particles are properly positioned in a corresponding two-dimensional or multidimensional scatter diagram.

The method 500 also includes setting a threshold (i.e., a gate) and removing S4 the invalid data to reduce the size of the calculation. The step of removing S4 the invalid data may be the same as that of the preceding embodiment.

The method 500 also includes calculating S6 the distance between cells or particles. If the distance between two cells or particles is zero, only one of the cells or particles is allowed to participate in the clustering analysis, but both cells or particles are counted. Thereafter, a distance matrix is formed from the calculated distances.

The method 500 also includes clustering S8 the cells or particles with a high degree of similarity into the same class. The classification may be performed using a hierarchical clustering method, a fast clustering method or another clustering method, such as fuzzy clustering, neural network clustering, etc.

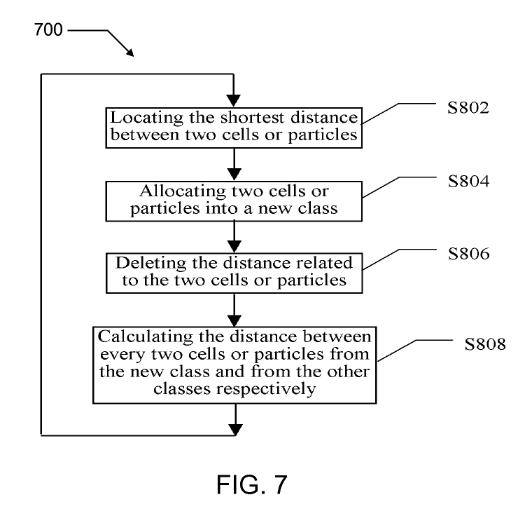

An example hierarchical clustering method 700 is illustrated in FIG. 7, and includes locating S802 the shortest distance between two cells or particles from the collection of distances as calculated. The smallest element on an off-diagonal line in the matrix D₍₀₎ is selected and denoted as dᵤᵥ.

The hierarchical clustering method 700 also includes allocating S804 the above two cells or particles into a new class having the same dimensions. That is, eᵤ and eᵥ are grouped to form a new class Gᵣ={eᵤ, eᵥ}. The method 700 further includes deleting S806 the distance related to the two cells or particles from the collection of distances. That is, the columns and rows corresponding to eᵤ and eᵥ are deleted from D₍₀₎. The method 700 also includes calculating S808 a distance between cells or particles from the new class Gᵣ, and from the other classes respectively. This distance is added into the collection to obtain a new distance matrix D₍₁₎. From D₍₁₎, the above-mentioned steps are repeated to obtain D(₂), etc. until m events are clustered into a major class.

In one embodiment, the method 700 includes calculating S808 the distance between cells before deleting S806 the distance related to the two cells or particles. Each clustering is assigned a number, and the level (i.e., distance) of two classes is recorded during clustering. A clustering hierarchical diagram is then plotted.

Returning to FIG. 5, the method 500 also includes classifying S10 data according to characteristics of the sample. The data may be divided into different classes at different hierarchical levels in the clustering hierarchical diagram. Because it is possible to know how many classes of particles the sample should have under a certain measuring principle based on the characteristic of the sample, the corresponding number of classes may be obtained by selecting the hierarchical level.

However, a certain subclass of the sample may have a poor consistency in terms of the characteristics due to individual differences among the samples. That is to say, the particles in this class are relatively disperse, or the difference between a first subclass and a second subclass is not evident (i.e., the distance is short). In this case, an error occurs if data are still divided into g classes, which reduces the credibility of the classification result. Therefore, the method 500 may also include evaluating S12 the clustering effect after classifying S10 the data according to the characteristics of the sample.

In one embodiment, evaluating S12 the classification effect includes calculating parameters concerning the clustering effect corresponding to integers from 1 to L+r, where L is the number of classes that should be contained in the sample (determined based on the measuring principle, and is an integer larger than or equal to 1), and r is an empirically determined integer larger than 0.

If there are r total classes at a certain hierarchical level (i.e., a distance), the sum of squares of dispersions in class Gₖ is:

$${S}_{k} = {\sum }_{i ∈ {G}_{k}} {( {x}_{i} - {\overline{x}}_{k} )}^{T} ( {x}_{i} - {\overline{x}}_{k} ) ,$$

where xᵢ is a vector (xᵢ₁, xᵢ₂, . . . xᵢₚ)^{T} of an event eᵢ, T represents transposition of the matrix, and x̅ₖ is a center of gravity of class Gₖ (e.g., the center of gravity of all events participating in the calculation in class Gₖ, whose coordinates are the mean of altitudes of the event, and the smaller the Sₖ, the more similar the events in Gₖ).

Defining

$${P}_{g} = {\sum }_{k = 1}^{G} {S}_{k} ,$$

the sum of squares of dispersions of all events is:

$$T = {\sum }_{i = 1}^{m} {( {x}_{i} - \overline{x} )}^{T} ( {x}_{i} - \overline{x} ) .$$

A pseudo-F statistic quantity PSF represents the effect of dividing all data into g classes:

$$PSF = \frac{( T - {P}_{g} ) / ( g - 1 )}{{P}_{g} / ( m - g )} ,$$

where m is the total number of events participating in the calculation in the distance matrix, in which a larger PSF indicates that these events can be divided into g classes significantly.

If the sample has L classes under a certain measuring principle, PSF values corresponding to the classes numbered from 1 to L+r (r>0) is calculated, where r is usually 3˜5.

Evaluating S12 the classification effect also includes locating the integer corresponding to the largest parameter concerning the clustering effect. If the largest PSF occurs when the events are divided into q classes, q classes are considered the most suitable.

As above mentioned, q is not equal to L in most cases. Therefore, the method 500 includes querying S14 whether or not the classification is reasonable. Specifically, the integer q located in step S2 is compared with the number L of classes. The method 500 includes outputting S16 the classification result if the classification is reasonable, or proceeding S18 to an abnormal sample processing program if the classification is unreasonable.

The following two situations generally occur when classification is reasonable:

(i) when q>L, the number q of classes is considered, and an alarm notifies that a new class is present (usually abnormal cell population), and the process goes to an abnormal sample processing program. The abnormal sample processing program counts the cells or particles in L classes and calculates, for example, the percentages in the L classes only. The new class does not participate in the calculation of the percentage, but needs to be treated by a fixed borderline.

(ii) when (L−o)<q≦L, the number L of classes is considered, the calculation is done as normal, and the classification result is outputted S16. The value of o is an empirical value depending on a large amount of samples. There are q classes only in the sample, and data concerning the other classes is zero.

When q≦(L−o) (which means that this sample is abnormal and the classes cannot be distinguished), no calculation is carried out, and an alarm is raised to proceed S18 the method 500 to an abnormal sample processing program. In this case, this means that a fault is present in the instrument, or that leukemia or a reagent becomes inoperative on the blood.

In addition to the above-mentioned pseudo-F statistic quantity, an R2 statistic quantity, a half-deflection correlated statistic quantity or a pseudo-t2 statistic quantity, etc. can also be adopted for evaluating the classification effect.

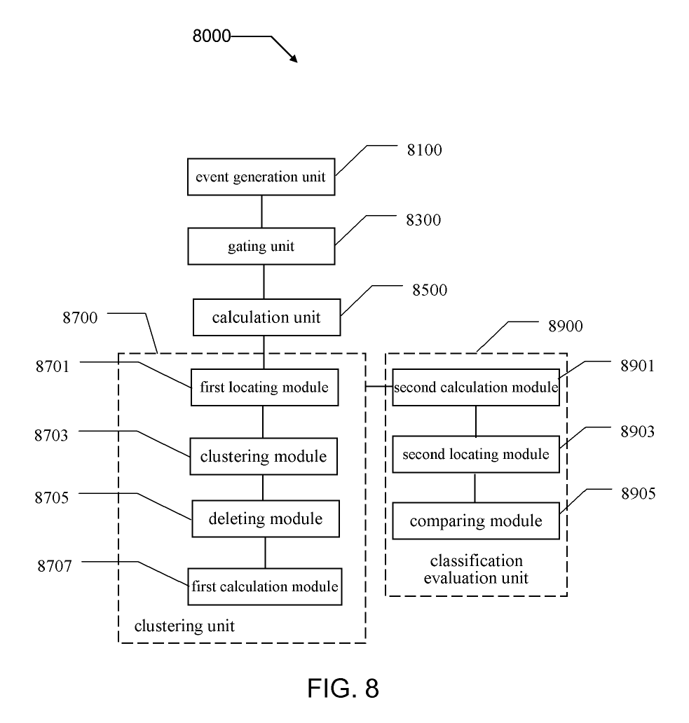

FIG. 8 shows a classification device 8000 (corresponding to the analysis module as shown in FIG. 4) based on flow cytometry for realizing the above-mentioned method. The classification device 8000 includes an event generation unit 8100, a calculation unit 8500 and a clustering unit 8700. The event generation unit 8100 characterizes each measured cell or particle as a vector that is at least two-dimensional and is associated with the intensity of optical signals in various paths thereof based on the at least two-path optical signals generated when the cells or particles are passing through an irradiated area one by one. The calculation unit 8500 calculates a distance between effective cells or particles based on the vector generated by the event generation unit 8100. The shorter the distance, the higher the similarity between two cells or particles. The clustering unit 8700 clusters the cells or particles with high similarity into the same class. The clustering unit 8700 may perform multiple iterations of clustering until the effective cells or particles are clustered into a proper number L of classes that should be contained in a sample, which is determined based on a measuring principle. In another embodiment, the clustering unit 8700 clusters the effective cells or particles into one class only.

To reduce the data involved in the calculation and improve the efficiency of classification, the classification device 8000 further includes a gating unit 8300 for setting a threshold to delete data which do not meet the criterion of the threshold.

The clustering unit 8700 further includes a first locating module 8701 for locating the shortest distance between two cells or particles among a collection of distances, a clustering module 8703 for grouping said two cells or particles into a new class having the same dimensions, a deleting module 8705 for deleting the distance related to the two cells or particles from the distance collection, and a first calculation module 8707 for calculating a distance between cells or particles from the new class and from the other classes, respectively, and adding the distance into the distance collection.

The classification device 8000 further includes a classification evaluation unit 8900 for evaluating the clustering effect to determine a correct number of classes which should be contained in a sample.

The classification evaluation unit 8900 further includes a second calculation module 8901 for calculating parameters concerning the clustering effects corresponding to integers from 1 to L+r, where L denotes the number of classes which should be contained in a sample and is determined based on a measuring principle, and is an integer larger than or equal to 1. Further, r is an empirically determined integer larger than 0. The classification evaluation unit 8900 further includes a second locating module 8903 for locating an integer q corresponding to the biggest parameter concerning the clustering effect, and a comparing module 8905 for comparing the integer q located by the second locating module 8903 with the number L of classes. If q>L, the comparing module 8905 takes q as the number of classes the sample should have. If L−o<q≦L, the comparing module 8905 takes L as the number of classes. If q≦L−o, the classification and calculation terminate.

The parameter concerning the clustering effect calculated by the second calculation module 8901 is a pseudo-F statistic quantity. The second calculation module 8901 further includes a third calculation module (not shown) for calculating the sum of squares of dispersions in each class according to the formula

$${S}_{k} = {\sum }_{i ∈ {G}_{k}} {( {x}_{i} - {\overline{x}}_{k} )}^{T} ( {x}_{i} - {\overline{x}}_{k} ) ,$$

where Sₖ is the sum of squares of dispersions in class Gₖ, xᵢ is a vector (xᵢ₁, xᵢ₂, . . . xᵢₚ)^{T} of the i^{th} cell in class Gₖ, and x̅ₖ is a center of gravity of class Gₖ. A fourth calculation module (not shown) calculates the sum P_{g} of the sums of squares of dispersions of all classes when the sample is divided into g classes. A fifth calculation module (not shown) calculates a pseudo-F statistic quantity when the sample is divided into g classes based on the formula

$$P S F = \frac{( T - {P}_{g} ) / ( g - 1 )}{{P}_{g} / ( m - g )} .$$

The following is an example embodiment of a blood cell analyzer.

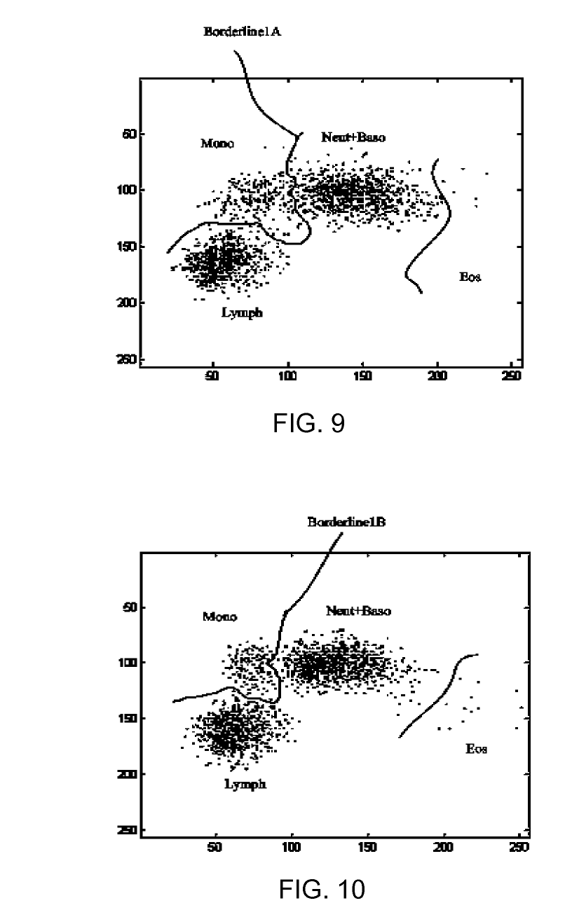

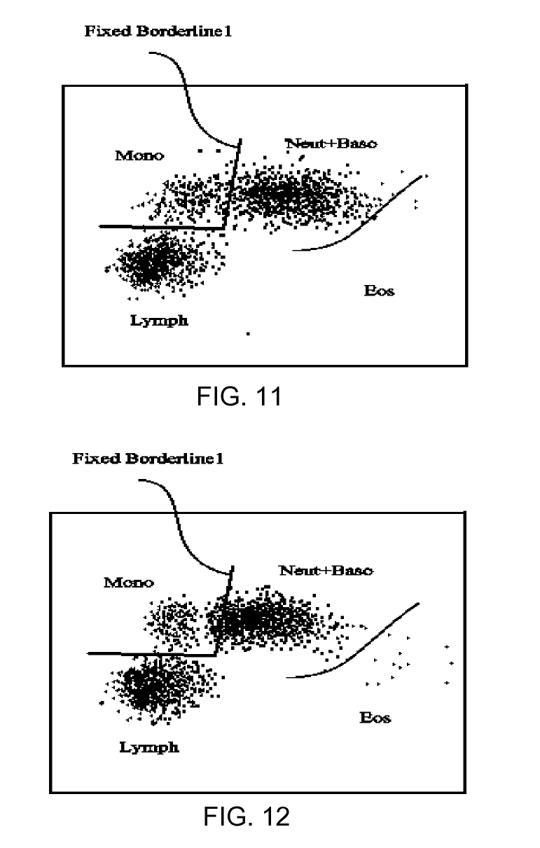

In a flow cytometry-based blood cell analyzer, the white blood cells in whole blood are divided into four subclasses using FSC and SSC, including lymphocyte (Lymph), monocyte (Mono), neutrophil (Neut), basophil (Baso) and acidophi (Eos). FIGS. 9 and 10 show the results of classifying two different samples A and B according to the embodiments of the present disclosure. As shown in FIGS. 9 and 10, different Borderline1A and Borderline1B are generated from different sample data for the classification. In the prior art, sample data are classified using fixed borderlines on the scatter diagram formed by two-dimensional signals. However, the fixed borderlines cannot reflect the individual differences among the samples. FIGS. 11 and 12 each show a classification using a Fixed Borderline 1. It can be seen that part of the Neut cells in the sample B are assigned to Mono cells as the result of the fixed borderline, which causes a deviated result. The classification method according to the embodiments of the present disclosure automatically adjust the borderlines for classification according to different samples, which makes the classification result more reasonable.

One of the advantages achieved by the method or device according to the embodiments of the present disclosure is to carry out a clustering calculation whenever a sample is measured, which is a method of automatically classifying any sample. That is, classification is different with a different sample, i.e., it has a self-adaptability for different samples. However, a conventional method carries out classification using a fixed borderline, so a significant dispersion occurs when a sample does not meet the common characteristics of the fixed borderlines.

Though U.S. Pat. No. 5,627,040 teaches that the position of the borderline for each class of particles can be flexible, the shape, size and azimuth of the borderline for classification are fixed, which still does not address the above-mentioned problem.

Although there is provided an automatic classification algorithm in U.S. Pat. No. 6,944,338, this algorithm is based on a two-dimensional square matrix, in which many ineffective data points are included in the calculation. Additionally, when effective data is too dispersive, the calculation effect is reduced drastically, because the point located outside either a big group or a small group is considered as a separate class. However, this may not true.

Another advantage of the method or device according to the embodiments of the present disclosure is that the algorithm is based on data, instead of a drawing or an image, which allows classification of multidimensional data. However, U.S. Pat. No. 6,944,338 discloses a technique only directed to two-dimensional data. The commonly used prior art methods for dividing borderlines in a scatter diagram is only effective for three-dimensional data at the most.

A person of ordinary skill in the art will recognize that the described features, operations, or characteristics disclosed herein may be combined in any suitable manner in one or more embodiments. It will also be readily understood that the order of the steps or actions of the methods described in connection with the embodiments disclosed may be changed as would be apparent to those skilled in the art. Thus, any order in the drawings or Detailed Description is for illustrative purposes only and is not meant to imply a required order, unless specified to require an order.

Embodiments may include various steps, which may be embodied in machine-executable instructions to be executed by a general-purpose or special-purpose purpose computer (or other electronic device). Alternatively, the steps may be performed by hardware components that include specific logic for performing the steps or by a combination of hardware, software, and/or firmware.

Embodiments may also be provided as a computer program product including a machine-readable medium having stored thereon instructions that may be used to program a computer (or other electronic device) to perform processes described herein. The machine-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/machine-readable medium suitable for storing electronic instructions.

As used herein, a software module or component may include any type of computer instruction or computer executable code located within a memory device and/or transmitted as electronic signals over a system bus or wired or wireless network. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

It will be understood by those having skill in the art that many changes may be made to the details of the above-described embodiments without departing from the underlying principles of the invention. The scope of the present invention should, therefore, be determined only by the following claims.

What is claimed is:

1. An automatic classification method for flow cytometry, comprising:
characterizing cells or particles as a vector that is at least two-dimensional and associated with an intensity of optical signals in various paths thereof, based on at least two-path optical signals generated when the cells or particles are passing through an irradiated area one by one, wherein at least some characteristics of each cell or particle are represented as a respective multi-dimensional event vector;
calculating a distance between the event vectors, in which a shorter distance indicates a higher degree of similarity between two cells or particles; and
clustering the cells or particles with a high degree of similarity into the same class until the effective cells or particles are clustered into a number L of classes that should be contained in a sample and is determined based on a measuring principle.

2. The automatic classification method for flow cytometry according to claim 1, further comprising:
setting a threshold to delete data of the cells or particles that do not meet a criterion of the threshold.

3. The automatic classification method for flow cytometry according to claim 1, wherein the calculated distance is determined based on at least one of Euclidean distance, absolute distance, Minkowski distance, Chebyshev distance, weighted variance distance, and Markov distance.

4. The automatic classification method for flow cytometry according to claim 3, wherein clustering comprises adopting a hierarchical clustering method comprising:
locating the shortest distance between two cells or particles in a collection of distances as calculated;
clustering the two cells or particles into a new class with the same dimensions;
deleting the distance related to the two cells or particles from the distance collection; and
calculating a distance between the cells or particles in the new class and in the other classes, respectively, and adding the distance into the collection of distance.

5. The automatic classification method for flow cytometry according to claim 4, wherein locating the shortest distance between two cells or particles in the collection of distances, when the distance between the two cells or particles is zero, allowing only one of these two cells or particles to participate in clustering analysis, and recording both cells or particles when counting.

6. The automatic classification method for flow cytometry according to claim 4, further comprising:
assigning a number and level to each clustering of the two cells or particles; and
recording the assigned number and level during the course of clustering.

7. The automatic classification method for flow cytometry according to claim 1, wherein the effective cells or particles are finally clustered into one class.

8. The automatic classification method for flow cytometry according to claim 7, further comprising:
evaluating clustering effects to determine a correct number of classes which should be contained in a sample.

9. The automatic classification method for flow cytometry according to claim 8, further comprises:
calculating parameters about the clustering effect corresponding to integers from 1 to L+r respectively, where L is a number of classes which should be contained in a sample and determined based on the measuring principle and is an integer larger than or equal to 1, and wherein r is an empirically determined integer larger than 0;
locating an integer q corresponding to the biggest parameter about the clustering effect;
comparing the integer q with the number L of classes, wherein if q>L, the number of classes in the sample is q, wherein if L−o<q≦L, L is the number of classes in the sample, and wherein if q≦L−o, classification and calculation terminate.

10. The automatic classification method for flow cytometry according to claim 9, wherein the parameter about clustering effect is a pseudo-F statistic quantity, and wherein calculating the pseudo-F statistic quantity comprises:
calculating a sum of squares of dispersions in each class according to the formula
$${S}_{k} = {\sum }_{i ∈ {G}_{k}} {( {x}_{i} - {\overline{x}}_{k} )}^{T} ( {x}_{i} - {\overline{x}}_{k} ) ,$$
where Sₖ is the sum of squares of dispersions in class Gₖ, and xᵢ is a vector (xᵢ₁, xᵢ₂, . . . xᵢₚ)^{T} of the i^{th} cell or particle in class Gₖ, and x̅ₖ is a center of gravity of class Gₖ;
calculating sum P_{g} of the sums of squares of dispersions of all classes when the sample is divided into g classes; and
calculating the pseudo-F statistic quantity PSF based on the formula
$$P S F = \frac{( T - {P}_{g} ) / ( g - 1 )}{{P}_{g} / ( m - g )}$$
where the sample is divided into g classes.

11. An automatic classification device for flow cytometry, comprising:
an event generation unit for characterizing cells or particles as a vector that is at least two-dimensional and associated with an intensity of optical signals in various paths based on at least two-path optical signals generated when the cells or particles are passing through an irradiated area one by one;
a calculation unit for calculating a distance between the cells or particles based on the vector generated by the event generation unit, in which a shorter distance indicates a higher degree of similarity between two cells or particles; and
a clustering unit for clustering the cells or particles with a high degree of similarity into the same class until the effective cells or particles are clustered into a number L of classes that should be contained in a sample based on a measuring principle.

12. The automatic classification device for flow cytometry according to claim 11, further comprising:
a gating unit for setting a threshold to delete data of the cells or particles that do not meet a criterion of the threshold.

13. The automatic classification device for flow cytometry according to claim 11, wherein the calculated distance between is determined based on at least one of Euclidean distance, absolute distance, Minkowski distance, Chebyshev distance, Weighted Variance distance, and Markov distance.

14. The automatic classification device for flow cytometry according to claim 13, wherein the clustering unit comprises:
a first locating module for locating the shortest distance between two cells or particles in a collection of all distances as calculated;
a clustering module for clustering the two cells or particles into a new class with the same dimensions;
a deleting module for deleting the distance related to the two cells or particles from the distance collection; and
a first calculation module for calculating a distance between the cells or particles in the new class and in the other classes, respectively, and adding the distance into the distance collection.

15. The automatic classification device for flow cytometry according to claim 11, wherein the clustering unit clusters the effective cells or particles into one class.

16. The automatic classification device for flow cytometry according to claim 15, further comprising:
a classification evaluation unit for evaluating a clustering effect to determine a correct number of classes that should be contained in a sample.

17. The automatic classification device for flow cytometry according to claim 16, wherein the classification evaluation unit further comprises:
a second calculation module for calculating parameters about the clustering effects corresponding to integers from 1 to L+r respectively, where L is a number of classes that should be contained in a sample and is determined based on the measuring principle, and is an integer larger than or equal to 1, and wherein r is an empirically-determined integer larger than 0;
a second locating module for locating an integer q corresponding to the biggest parameter about the clustering effect;
a comparing module for comparing the integer q located by the second locating module with the number L of classes, wherein if q>L, q is taken as the number of classes in the sample, wherein if L−o<q≦L, L is taken as the number of classes in the sample, and wherein if q≦L−o, classification and calculation terminate.

18. The automatic classification device for flow cytometry according to claim 17, wherein the parameter about clustering effect calculated by the second calculation module is a pseudo-F statistic quantity, and wherein the second calculation module further comprises:
a third calculation module for calculating a sum of squares of dispersions in each class according to the formula
$${S}_{k} = {\sum }_{i ∈ {G}_{k}} {( {x}_{i} - {\overline{x}}_{k} )}^{T} ( {x}_{i} - {\overline{x}}_{k} ) ,$$
where Sₖ is sum of squares of dispersions in class Gₖ, and xᵢ is a vector (xᵢ₁,xᵢ₂, . . . xᵢₚ)^{T} of the i^{th} cell or particle in class Gₖ, and x̅ₖ is a center-of-gravity of class Gₖ;
a fourth calculation module for calculating sum P_{g} of the sums of squares of dispersions of all classes where the sample is divided into g classes; and
a fifth calculation module for calculating a pseudo-F statistic quantity PSF based on the formula
$$P S F = \frac{( T - {P}_{g} ) / ( g - 1 )}{{P}_{g} / ( m - g )} ,$$
where the sample is divided into g classes.

19. An automatic classification and statistics system for flow cytometry, comprising:
a sample generation device including a gas-liquid transmission controlling module and a flow chamber, which are connected to each other, the gas-liquid transmission controlling module configured to pass a sample fluid comprising cells or particles to be measured and encased by sheath fluid through the flow chamber;
an irradiation device for emitting a light beam to irradiate the sheath fluid passing through the flow chamber;
a detector for collecting at least two-path optical signals generated when the cells or particles are passing through an irradiated area one by one; and
a processor for classification and statistics for:
characterizing the cells or particles as a vector which is at least two-dimensional and associated with an intensity of optical signals in various paths thereof based on the optical signals collected by the detector;
calculating a distance between effective cells or particles, in which a shorter distance indicates a higher degree of similarity between two cells or particles; and
clustering the cells or particles with a high degree of similarity into the same class for multiple times until at least the effective cells or particles are clustered into a number L of classes that should be contained in a sample and is determined based on a measuring principle.

20. The automatic classification and statistics system for flow cytometry according to claim 19, wherein the processor for classification and statistics is further configured to set a threshold before calculating the distance between the effective cells or particles to delete data of the cells or particles which do not meet a criterion of the threshold.

21. The automatic classification and statistics system for flow cytometry according to claim 20, wherein the processor for classification and statistics is further configured to cluster the effective cells or particles into one class.

22. The automatic classification and statistics system for flow cytometry according to claim 21, wherein the processor for classification and statistics is further configured to calculate parameters about the clustering effects corresponding to integers from 1 to L+r, locate an integer q corresponding to the biggest parameter about the clustering effect, and compare the located integer q with the number L of classes, wherein if q>L, q is the number of classes in the sample; wherein if L−o<q≦L, L is the number of classes in the sample; and wherein if q<L−o, classification and calculation terminate, L denoting a number of classes which should be contained in the sample and that is determined based on the measuring principle and being an integer larger than or equal to 1, and r denoting an empirically determined integer larger than 0.

23. A computer-readable medium comprising program code for performing a method for flow cytometry, the method comprising:
characterizing cells or particles as a vector that is at least two-dimensional and associated with an intensity of optical signals in various paths thereof, based on at least two-path optical signals generated when the cells or particles are passing through an irradiated area one by one, wherein at least some characteristics of each cell or particle are represented as a respective multi-dimensional event vector;
calculating a distance between the event vectors, in which a shorter distance indicates a higher degree of similarity between two cells or particles; and
clustering the cells or particles with a high degree of similarity into the same class until the effective cells or particles are clustered into a number L of classes that should be contained in a sample and is determined based on a measuring principle.

24. An apparatus for flow cytometry, comprising:
means for characterizing cells or particles as a vector that is at least two-dimensional and associated with an intensity of optical signals in various paths thereof, based on at least two-path optical signals generated when the cells or particles are passing through an irradiated area one by one, wherein at least some characteristics of each cell or particle are represented as a respective multi-dimensional event vector;
means for calculating a distance between the event vectors, in which a shorter distance indicates a higher degree of similarity between two cells or particles; and
means for clustering the cells or particles with a high degree of similarity into the same class until the effective cells or particles are clustered into a number L of classes that should be contained in a sample and is determined based on a measuring principle.
